(19) United States
(12) Patent Application Publication
(10) Pub. No.: US 2010/0137805 A1
(43) Pub. Date: Jun. 3, 2010

(54) APPARATUS FOR SECURING AN INVASIVE TUBE TO A BODY

(76) Inventors: Samuel Farchione, Sarasota, FL (US); Shari M. Rincon, Sarasota, FL (US); Thomas Farchione, Sarasota, FL (US)

Correspondence Address: Gifford, Krass, Sprinkle, Anderson & Citkowski,;P.C., 2701 Troy Center Drive, Suite 330, Post Office Box 7021, Troy, MI, 48007-7021, US

(21) Appl. No.: 12/470,266

(22) Filed: May 21, 2009

Related U.S. Application Data
(60) Provisional application No. 61/055,060, filed on May 21, 2008.

(51) Int. Cl. A61M 25/02

(52) U.S. Cl. 604/179

(57) ABSTRACT

An apparatus is provided for securing an elongated member such as a feeding tube, catheter or similar invasive tube, wire, cable or cord to a person's body. The apparatus may include a belt having an aperture that extends through the belt, and a securable flap that may cover the aperture. Absorbent pads may be positioned proximate the aperture on the interior of the belt. Pads or cushions may also be provided within or on the belt to make the belt more comfortable for the user to wear.

### CROSS-REFERENCE TO RELATED APPLICATIONS

The present application claims priority from U.S. Provisional Application No. 61/055,060 filed May 21, 2008.

### FIELD OF THE INVENTION

Disclosed is an apparatus for securing an elongated member such as a feeding tube, catheter or similarly invasive tube, wire, cable or cord to a person's body.

### BACKGROUND OF THE INVENTION

Percutaneous endoscopic gastrostomy (PEG) is a generally safe and effective way of providing food, liquids and medication directly into a patient's stomach. The procedure, which is usually performed on patients that have difficulty swallowing, involves a physician placing an endoscope into a patient's mouth. The endoscope is then advanced through the patient's esophagus and into the stomach where it is used to ensure correct installation and positioning of a PEG tube (i.e., a feeding tube) into the patient's stomach. Once installed, the PEG tube rests in the patient's stomach and exits through an incision in the abdomen.

A PEG tube, therefore, allows a user to meet his or her nutritional needs. However, while the use of a PEG tube may be necessary in some cases, it can also be debilitating for the user. A tube may, for example, become dislodged during sleep or accidentally removed when snagged on people and things (e.g., wheelchair) in the vicinity of the user. The danger or fear of an accidental removal of a tube may also inhibit the ability of a user to participate in physical activities such as golf, exercise, walking, running and dancing. The incision in the abdomen surrounding the tube may leak, irritating and burning the user's skin and damaging the user's clothing. The adhesive tape(s) that is used to secure the PEG tube to the user may also irritate the user's skin.

An apparatus for securing a catheter tube to a body is disclosed by U.S. Pat. No. 5,403,285. The apparatus included an elongated, flexible, elastic member that is adapted to extend completely around the chest of a person and over a flexible tube that exits the person's body. A flap is provided on the elastic member having hook and loop fasteners for selectively covering up the coiled tube when not in use.

U.S. Pat. No. 5,048,512 disclosed a gastrostomy tube protector belt. The belt incorporated a pocket having a closure in which a gastrostomy tube can be stored and protected.

### SUMMARY OF THE INVENTION

The present application claims priority from U.S. Provisional Application No. 61/055,060 filed May 21, 2008, the entire disclosure of which is incorporated herein by reference.

Disclosed is an apparatus for securing an invasive medical tube (e.g., a feeding tube, catheter, or the like) to a body. The apparatus may, for example, be used in connection with a feeding tube, a j-tube (jejunostomy), a PEG tube, a gastronomy tube or the like.

The apparatus may include a length of flexible material having a pair of ends. A fastener may be provided on the material proximate each end and there may be an aperture that extends through the material. The fasteners may be removably secured together so that the material may be configured into a belt that may be worn by a user. This belt, for example, may be worn so that the aperture is positioned in-line with a user's incision/feeding tube and so that the tube may pass through the aperture.

The apparatus may further include an anchor tab that has one end secured to the flexible material and an opposite end that may be detachably secured to the flexible material through, for example, the use of fasteners on the tab and the material. The anchor tab may be positioned on the flexible material so that the tab can be extended across the aperture and thereby limit the travel of any tube that passed though the aperture.

A cover or flap may also be provided that can overlay and protect the aperture and the anchor tab and the tube from exposure. Moreover, when placed in a down or closed position, the flap may also function as a guide and/or holder for the tube.

### BRIEF DESCRIPTION OF THE DRAWINGS

Reference will now be had to the attached drawings wherein like reference numerals refer to like parts throughout, and wherein:

FIG. 1 is an front perspective view of an embodiment of the apparatus being worn by a user;

FIG. 2A is an front perspective view of an embodiment of the apparatus of FIG. 1 being worn by a user and showing the flap or cover in a raised or open position;

FIG. 2B is an enlarge view of the FIG. 2A, but showing the anchor tab in the a raised or open position;

FIG. 3 is an front perspective view of an embodiment of the apparatus being worn by a user with the user shown in phantom such that bumper pads are visible;

FIG. 4 is an rear perspective view of an embodiment of the apparatus being worn by a user with the user shown in phantom (reverse from FIG. 3) such that absorbent pad is visible.

FIG. 5 is a planar side view of the apparatus of FIG. 1 showing an elastic section of the belt;

FIG. 6 is an front perspective view of an embodiment of the apparatus of FIG. 1 being worn by a user and showing the fasteners for the belt; and

### DETAILED DESCRIPTION OF THE PREFERRED EMBODIMENTS

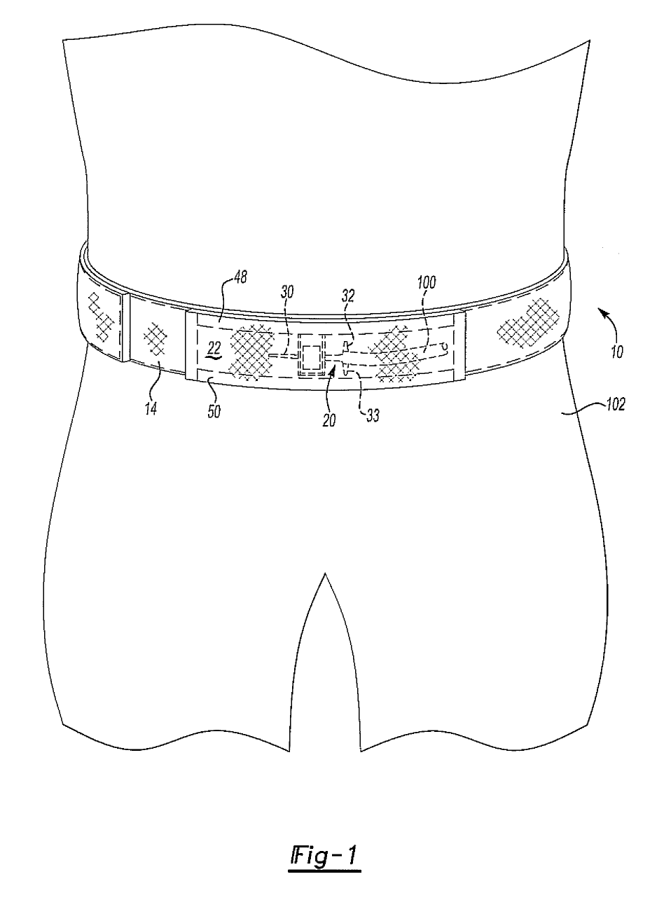

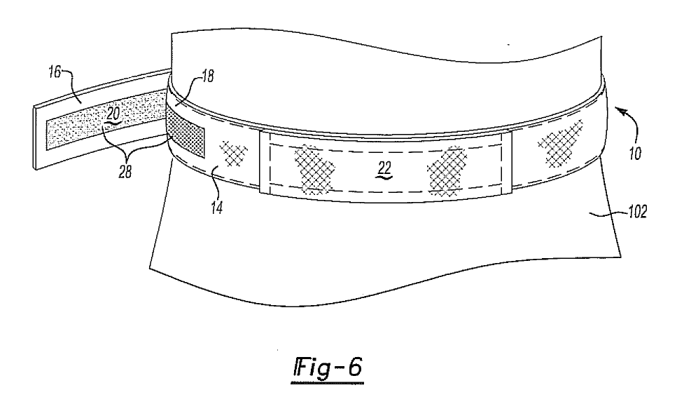

Referring now to FIGS. 1-6, one embodiment of an apparatus for securing an elongated member such as an invasive medical tube 100 (e.g., a feeding tube, catheter, tube, wire, cable, cord or the like) to a (non-human) or human body 102 may include an elongated belt 10 or loop. The belt 10 may be formed from a length of a flexible material and have an interior surface 12 or face, an exterior surface 14 or face, and a first 16 and a second end 18. An aperture 20 may be defined in, and extend through, the belt 10 from one surface 12 to the other 14 and a flap 22 or cover may be positioned on the exterior surface 14 proximate the aperture 20 so that it may overlay or cover the aperture 20 and secure/guide the tube 100. An absorbent pad 24 may be position on the interior surface 12 proximate the aperture 20 to collect fluid that may escape the tube 100 or an incision (not shown) in a user's body though which the tube 100 is inserted. “Bumper” pads 26 may also be fixably or removably positioned on the interior surface 12 to contact and cushion the user's back during use. These pads 26 may also be integrated into the belt 10 by positioning them between layers of material. The belt 10 may be secured around a user's body 102 using hoop and loop fasteners 28 that may be provided proximate each of the ends 16, 18.

Referring to FIGS. 1-4 and 6, the belt 10 may be constructed of 100% cotton fabric. However, other materials or blends of materials may also be used that have similar characteristics for comfort and long term wearability for a user. The belt 10 may be dimensioned to have a width of between 1 and 6 six inches and a corresponding length of between 1 and 6 feet. However, it will be appreciated that the belt 10 may also be custom designed and fitted to meet the specific size requirements of (human and non-human) users of all sizes and shapes.

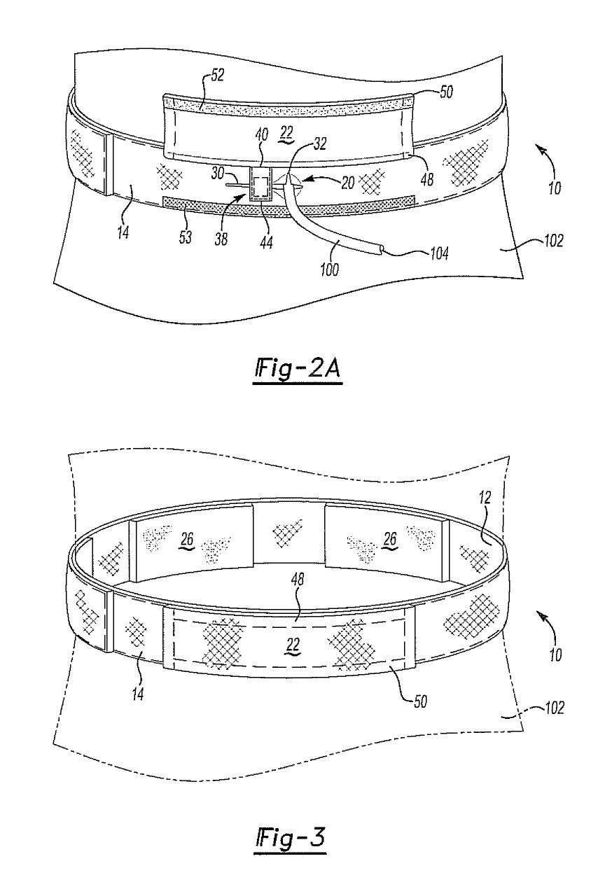

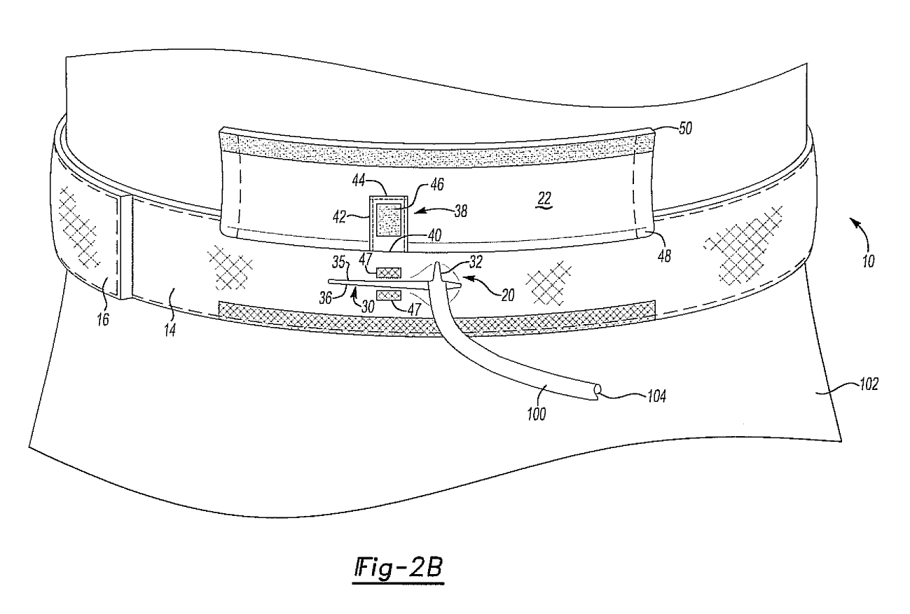

Still referring to FIGS. 1-4 and 6, the aperture 20 may include a lengthwise or longitudinal portion 30 the length of which may be dimensioned large enough to permit a user 102 to easily locate and pass an external end 104 of a tube 100 through the aperture 20. Accordingly, this longitudinal portion 30 may have a length between 0.3 and 4 inches. The aperture 20 may also include a widthwise or lateral portion or cut(s) that bisects the longitudinal portion 30. For example, as best shown in FIGS. 2A and 2B, the lateral portion of the aperture 20 may be configured to include opposed lateral cuts 32, 33 extending from respective sides 34, 35 of the longitudinal portion 30. In operation, this cross-cut or “button-hole” arrangement of the aperture 20 at the intersection of the longitudinal and lateral portions 30, 32 may function to help retain the tube 100 in position while allowing for small side-to-side and/or up-and-down movement of the tube 100.

Referring now to FIGS. 2A and 2B, an anchor tab 38 may be provided that is formed from the same or similar material as the belt 10. The tab 38, for example, may include one end 40 secured to the exterior surface 14 proximate the aperture 20, a body 42 having a sufficient length for the tab 38 to extended across the aperture 20, and an opposite end 44 that may be removably secured to the exterior surface 14 using hook and loop fasteners 46, 47 on the tab 38 and the exterior surface 14. The tab 38 may also be positioned nearer one end (i.e., the button-hole end) of the aperture 20 such that after the tube 100 is passed though the aperture 20, the travel of the tube 100 in aperture may be restrained by the placing the tab 38 in a down or closed position in which the opposite end 44 is secured to the exterior surface 14.

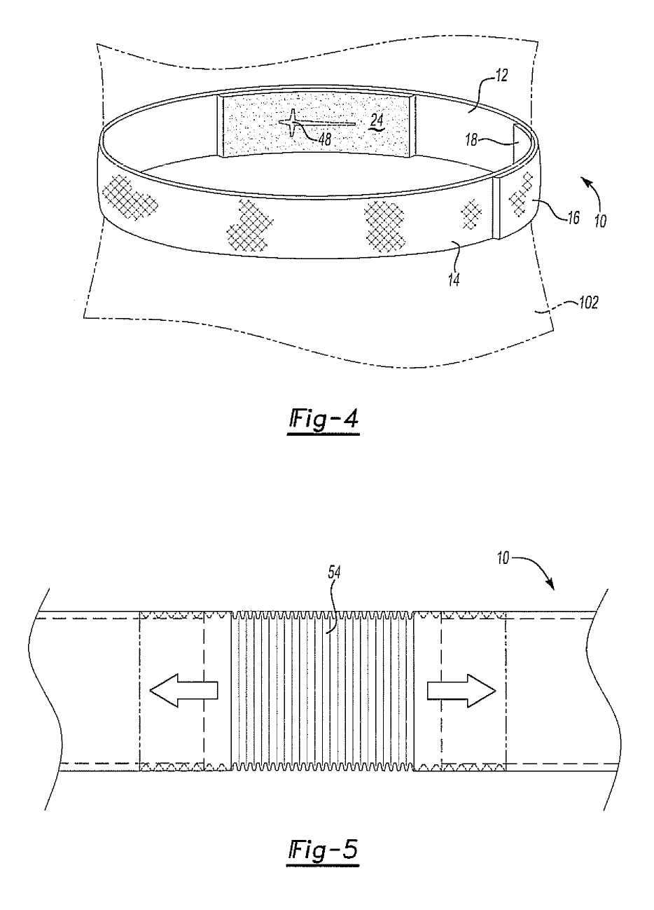

Referring now to FIG. 4, the absorbent pad 24 may be removably positioned on the interior surface 12 of the belt 10 using hook and loop fasteners or the like. The pad 24 may include a second aperture 48 that may be aligned with the aperture 20 of the belt 10. The pad 24 may also be configured so that is substantially identical in shape to the aperture 20 of the belt 10 and formed using absorbent materials known in the art for collecting blood and/or other body fluids (e.g., a feminine napkin or the like).

Referring now to FIGS. 1-4 and 6, and as best shown in FIG. 2, the flap 22 or cover may be constructed from the same material used to construct the belt 10; although different materials may also be used. As shown, the flap 22 may be stitched or otherwise secured along one of its sides 48 to the exterior surface 14. An opposite side 50 of the flap may be removably secured to the belt 10 using hook and loop fasteners 52, 53 on the flap 22 and the exterior surface 14. However, it will be appreciated that snaps, buttons or other devices may also be used to secure, or removably secure, the flap 22 to the belt 10.

Referring now to FIG. 3, the “bumper” pads 26 may be constructed from neoprene, foam, or the like. The pads 26 may be permanently fixed to the belt 10, but may also be made removable (using tape, hook and loop fasteners, etc), so that the pads 26 may be 1) cleaned separately from the belt 10 and/or 2) repositioned on along the interior surface 12 to a position that is of greatest comfort for the user 102.

Referring to FIGS. 1-6, and as best shown in FIG. 5, the belt 10 may include sections or lengths of an elastomeric material 54 (i.e., an elastic band) so that the belt 10 may comfortably move with the user 102 while also maintaining a snug fit around the user 102. This elastomeric material 54 may be integrated into the flexible material for the belt 10 or, alternative, the elastomeric material 54 may be included as intermediate sections of the belt 10. For example, as shown in FIG. 5, a section of elastic band may be sewn between, and thus connect, two section of other material to form the flexible material of the belt 10. It will be appreciated that the belt 10 may include more than one of these intermediate sections of elastomeric material. Additionally, or alternatively, the elastomeric material 54 may be integral with material of the belt 10, or it may over or underlay the material of the belt 10.

In operation, the belt 10 may comfortably secure a tube 100 in place during all manner of activities (including sporting activities), and while sleeping. The belt 10 may also mitigate against snagging of the tube. More specifically, in operation the belt 10 may be positioned around a user 102 with the tube 100 extending through the aperture 48 of the absorbent pad 24 and the aperture 20 of the belt 10. When not in use, the tube 100 may be enclosed and covered by the flap 22. However, access to the tube 100 may be had by simply lifting the flap 22.

Where the tube 100 is a feed tube, it may also be appreciated that the tube 100, and thus the aperture(s) 20, 48 may be positioned along the user's front, with the bumper pads 26 positioned so that they contact the user's back. However, as mentioned above, the belt 10, apertures 20, 48, and pads 26 may also be arranged around a user's body according to the actual needs of a particular user.

Having thus described my invention, other embodiments will become know to those of skill in the art that do not depart from the scope of the invention.

1. An apparatus for securing an elongated member to a user comprising:
a length of flexible material having a pair of ends, a fastener proximate each end, and defining an aperture that extends through the material, the fasteners being removably securable together to configure the length of material into a loop having an exterior surface and an interior surface;
an anchor tab having one end secured to the flexible material and an opposite end having a fastener operable to be detachably secured to the flexible material, and the anchor tab being positioned on the flexible material so that the tab is extendable across the aperture; and
a cover having a side secured to the flexible material and being operable to overlay the aperture and the anchor tab.

2. The apparatus of claim 1, further comprising an absorbent pad positioned on the flexible material proximate the aperture and on a surface of the flexible material opposite the anchor tab.

3. The apparatus of claim 1, further comprising a cushion positioned on the flexible material.

4. The apparatus of claim 1, wherein the length of flexible material comprises an elastomeric material.

5. The apparatus of claim 1, wherein the length of flexible material comprises at least two lengths of material and a length of elastomeric material is secured between the two lengths of material.

6. The apparatus of claim 1, further comprising a fastener positioned on the flexible material operable to be removably engaged by the fastener of the tab.

7. An apparatus for securing an elongated member to a user comprising:
a length of flexible material having a pair of ends, a fastener proximate each end, and defining an aperture that extends through the material and has a longitudinally extending portion and a pair of opposing lateral portions, the fasteners being secured together to configure the length of material into a loop; and
a cover having a side secured to the flexible material and being operable to overlay the aperture and the anchor tab.

8. The apparatus of claim 7 further comprising an anchor tab having one end secured to the flexible material and an opposite end having a fastener operable to be detachably secured to the flexible material, and the anchor tab being positioned on the flexible material so that the tab is extendable across the aperture; and

9. The apparatus of claim 8, further comprising an absorbent pad positioned on the flexible material proximate the aperture and on a surface of the flexible material opposite the anchor tab.

10. The apparatus of claim 8, further comprising a cushion positioned on the flexible material.

11. The apparatus of claim 8, wherein the length of flexible material comprises an elastomeric material.

12. The apparatus of claim 8 wherein the length of flexible material comprises at least two lengths of material and a length of elastomeric material is secured between the two lengths of material.

13. An apparatus for securing an elongated member to a user comprising:
a length of flexible material having a pair of ends, an elastic portion, a fastener proximate each end, and defining an aperture that extends through the material, the fasteners being secured together to configure the length of material into a loop;
an anchor tab having one end secured to the flexible material and an opposite end having operable to be detachably secured to the flexible material, the anchor tab being positioned on the flexible material so that the tab is extends across the aperture;
a cover having a side secured to the flexible material and being operable to overlay the aperture and the anchor tab.

14. The apparatus of claim 1, further comprising an absorbent pad positioned on the flexible material proximate the aperture and on a surface of the flexible material opposite the anchor tab.

15. The apparatus of claim 1, further comprising a cushion positioned on the flexible material.

16. The apparatus of claim 1, wherein the aperture comprises a longitudinally extending portion and a pair of opposing lateral portions and the anchor tab being positioned on the flexible material so that the tab extends across longitudinally extending portion of the aperture.
